(19) United States
(12) Patent Application Publication
(10) Pub. No.: US 2005/0058608 A1
(43) Pub. Date: Mar. 17, 2005

(54) Composition comprising at least one conductive polymer and at least one propellant, and process for the use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,335

(22) Filed: Jul. 16, 2004

Related U.S. Application Data
(60) Provisional application No. 60/492,292, filed on Aug. 5, 2003.

(30) Foreign Application Priority Data
Jul. 16, 2003 (FR) 03 08679

(51) Int. Cl. A61K 7/06; A61K 7/11; A61K 9/00

(52) U.S. Cl. 424/47; 424/70.17

(57) ABSTRACT

The present disclosure relates to a composition comprising, in a cosmetically acceptable medium, at least one propellant, and at least one conductive polymer. The at least one conductive polymer may, for example, comprise at least one repeating unit chosen from, for instance, aniline, pyrrole, thiophene, bisthiophene, furan, para-phenylene sulfide, para-phenylenevinylene, indole, aromatic amide, aromatic hydrazide, aromatic azomethine and aromatic ester radicals. The disclosure further relates to a process using such a composition, to give keratin fibers an optical effect.

This application claims benefit of U.S. Provisional Application No. 60/492,292, filed Aug. 5, 2003.

The present disclosure relates to a composition comprising, in a cosmetically acceptable medium, at least one conductive polymer and at least one propellant. The present disclosure also relates to a process for treating keratin fibers using the abovementioned composition, which may result in giving keratin fibers an optical effect.

The present disclosure further relates for example, to the field of treating keratin fibers, such as human keratin fibers, such as the hair.

Many of the treatments applied to keratin fibers, for instance, such as dyeing, bleaching or permanent-reshaping processes, can have major consequences on the characteristics of the fibers, for instance on their sheen. Thus, following repeated treatments, it is not uncommon to find that the treated fibers can become more or less dull, and continue to be so, despite the improvements that have been made in the processes used in the art.

In order to compensate for these negative effects, and in order to give the hair sheen, it is known practice to use, for example, lubricating hydrophobic substances, such as organic oils, waxes or silicones. However, the sheen effect obtained can lack intensity and can generally give the fibers an artificial look. Furthermore, these compositions can give the fibers an undesirable greasy or tacky feel.

Finally, the compounds of the prior art can have undesirable consequences when they are present in dye compositions. In essence, it has been found that the uptake of the dye may take place less effectively if the dye composition comprises these compounds. This may be reflected, for example, by weaker, less fast colorations.

Accordingly, one aspect of the present disclosure is thus to propose compositions that can give treated keratin fibers an appearance of sheen, without the drawbacks encountered with the standard known compositions.

Moreover, in certain cases, the composition according to the present disclosure may give color to the keratin fibers onto which it is applied.

An aspect of the present disclosure is thus a composition comprising, in a cosmetically acceptable medium:
(a) at least one propellant and
(b) at least one conductive polymer.

The present disclosure furthermore relates to a process for treating human keratin fibers, for example, the hair, with a composition comprising the at least one conductive polymer, which comprises applying the composition as disclosed herein to wet or dry fibers, and then drying the fibers or leaving them to dry.

Another aspect of the present disclosure is the use of a composition comprising at least one propellant and at least one conductive polymer, to give keratin fibers an optical effect.

For example, the composition according to the present disclosure uniformly gives the fibers a sheen that can be, for instance, more intense, more natural and more aesthetic than with the means of the prior art.

Moreover, when the at least one conductive polymer present in the composition as disclosed herein absorb in the visible spectrum, an optical effect, for instance sheen, and color are obtained simultaneously.

Finally, the fibers treated with the composition according to the present disclosure have a soft, non-greasy feel.

Other characteristics and benefits of the present disclosure will emerge more clearly upon reading the description and the example that follow.

In the text hereinbelow and unless otherwise indicated, the limits of a range of values are understood as forming part of that range.

For the purposes of the present disclosure, the term “optical effect” covers sheen, color, metallic, goniochromatic, moiré, fluorescent, thermochromatic and electromchromatic effects.

Moreover, for example, it should be noted that the sheen corresponds to the light intensity reflected at an angle α when the lock of hair is illuminated under an angle-α. The angle α conventionally used to measure this specular reflection, in other words the sheen, is equal to 200. This provision of sheen may be measured using a glossmeter as described, for example, in ISO standard 2813-1994 from AFNOR (August 1994, amended February 1997).

According to the present disclosure, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

For the purposes of the present disclosure, the expression “conductive polymer absorbing in the visible spectrum” means any conductive polymer having a non-zero absorbance in the wavelengths ranging from 400 to 800 nm, even if the absorption maxima of the polymer are outside this range.

The conductive polymers used in the context of the present disclosure are conductive polymers that are soluble or dispersible in the cosmetic medium suitable for use. The polymer is said to be soluble in the medium when it forms an isotropic clear liquid at 25° C. in a medium comprising water or a water/solvent mixture, this being obtained throughout all or part of a concentration ranging from 0.01% to 50% by weight of conductive polymer.

For example, the conductive polymers used in the context of the present disclosure may be conductive polymers that are soluble or dispersible in an aqueous medium, for instance, in water.

The polymer is said to be dispersible in the medium comprising water or a water/solvent mixture if, at 0.01% by weight, at 25° C., it forms a stable suspension of fine, generally spherical particles. The mean size of the particles comprising the dispersion is less than 1 μm, for example, ranging from 5 to 400 nm, such as from 10 to 250 nm. The particle sizes are measured by light scattering.

It should be noted, that these polymers do not require the use of a dispersant.

The conductive polymers can be, for example, in a form that is soluble in the medium of the composition.

Furthermore, the polymers may, for instance, have a conductivity ranging from 10⁻⁵ to 5×10⁵ siemens/cm, such as from 10⁻³ to 10⁵ siemens/cm, and from 10⁻¹ to 10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space (d), are applied by simple pressure to the sample to be analyzed. A current (I) is injected via the outer points using the current source, thus creating a variation in potential. The voltage (U) is measured between the two inner points connected to the voltmeter of the current generator.

In this configuration, the conductivity of the sample expressed in siemens (S) per centimetre, S/cm, is given by the following expression:
σ=(K×I)/(U×e)
wherein:
K is a coefficient depending on the position of the contacts on the surface of the sample. When the points are aligned and equidistant, K is equal to: π/log(2)
I: value of the injected current, expressed in amperes
U: the measured voltage value, expressed in volts
e: thickness of the sample, expressed in cm.

This expression can be used only when the thickness of the material is negligible compared with the distance (d) existing between two points (e/d<0.25). In order to obtain sufficiently small thicknesses and thus to be able to calculate the conductivity of the material, it is recommended to perform the measurement on a non-conductive support (for example a glass slide) coated with the material to be analyzed, obtained by evaporation of a dilute solution. In order to improve the homogeneity of the coating to be analyzed, it is also recommended to use the deposition technique known as spin coating.

According to one embodiment of the present disclosure, for example, the at least one conductive polymer comprised in the composition is chosen from polymers comprising at least one repeating unit chosen from
anilines of structure (I): 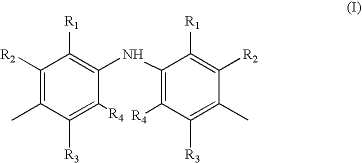
pyrroles of structures (IIa) and (IIb): 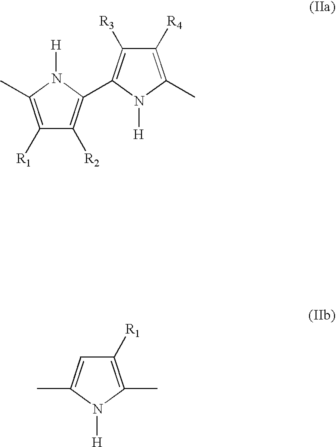
thiophenes or bisthiophenes of formulae (IIIa), (IIIb) and (IIIc): 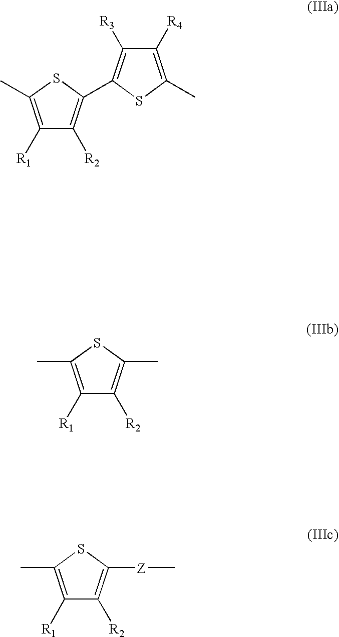
furans of formula (IV): 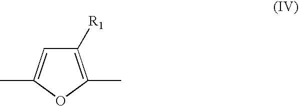
para-phenylene sulfides of formula (V): 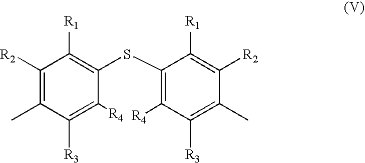
para-phenylenevinylenes of formula (VI): 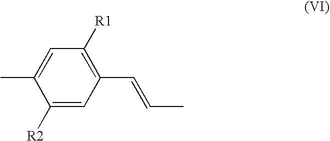
indoles of formula (VII): 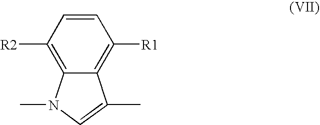
aromatic amides of formulae (VIIIa), (VIIIb), (VIIIc) and (VIIId): 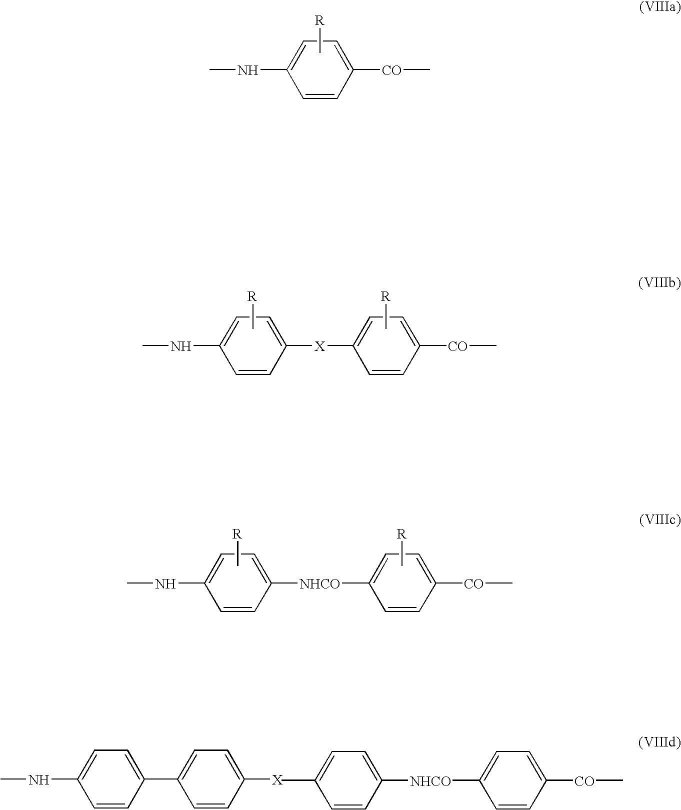
aromatic hydrazides of formulae (IXa), (IXb) and (IXc): 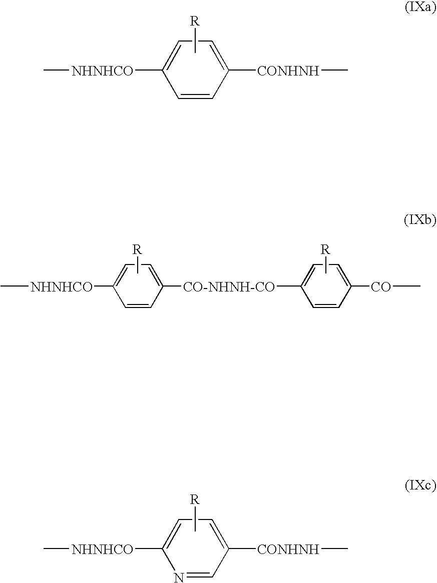
aromatic azomethines of formulae (Xa), (Xb) and (Xc): 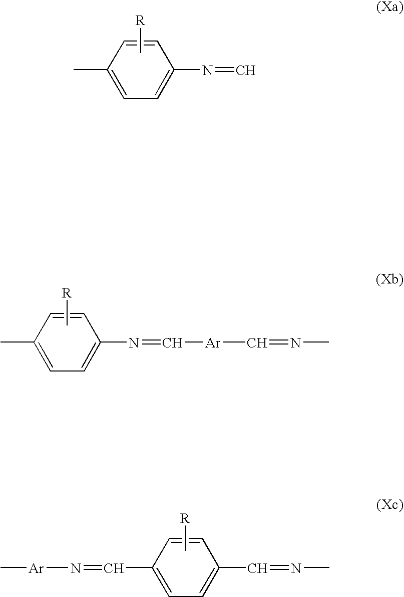
and aromatic esters of formulae (XIa), (XIb) and (XIc): 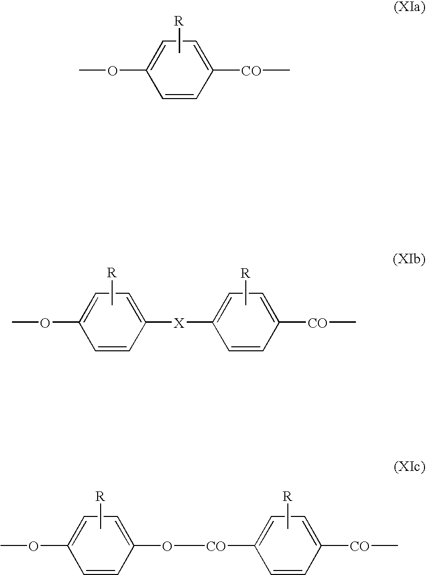
   wherein, in formulae (I) to (XI):
the radicals R, R₁, R₂, R₃, and R₄, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, and solubilizing groups;
Ar is a radical comprising a monoaromatic or polyaromatic radical;
X is chosen from oxygen and sulfur atoms, and —NHCO—, —SO₂—, —N═N—, —C(CH₃)₂—,
—CH₂—, —CH═CH—, and —CH═N— radicals; and
Z is chosen from —CH═CH— and —C≡C— radicals.

For example, Ar may be at least one radical chosen from the following:

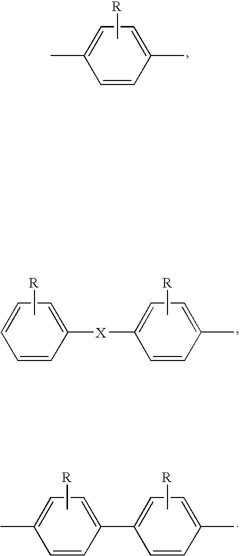

For the purposes of the present disclosure, the term “solubilizing group” means a group that ensures the dissolution of the molecule in the cosmetic medium, and such that the polymer has a conductive nature after drying the composition.

The at least one conductive polymer present in the composition as disclosed herein may comprise at least one repeating unit comprising at least one solubilizing group, and at least one other such unit lacking the solubilizing group.

Among the solubilizing groups that may be used as disclosed herein, non limiting mention may be made of, for example:
carboxylic (—COOH) and carboxylate (—COO-M+) radicals, wherein M is chosen from alkali metals, such as sodium or potassium, alkaline-earth metals, organic amines, such as primary, secondary or tertiary amines, alkanolamines, and amino acids,
sulfonic (—SO₃H) and sulfonate (—SO₃-M+) radicals, wherein M is as defined above,
primary, secondary and tertiary amine radicals,
quaternary ammonium radicals such as —NR₁₃+Z-, wherein Z is chosen from Br and Cl atoms, and (C₁-C₄)alkyl-OSO₃ radicals, and R′, which may be identical or different, are chosen from linear and branched C₁ to C₂₀ alkyl radicals, or two R′ may form a heterocycle with the nitrogen,
hydroxyl radicals, and
poly((C₂-C₃)alkylene oxide) radicals.

The carboxylic and sulfonic acid functional groups may optionally be neutralized with a base, such as sodium hydroxide, 2-amino-2-methylpropanol, triethylamine or tributylamine, for example.

The amine radicals may optionally be neutralized with a mineral acid, such as hydrochloric acid, or with an organic acid, such as acetic acid or lactic acid, for example.

In addition, it is also possible for the solubilizing radicals to be connected to the ring via a spacer group, for instance a radical chosen from —R″-, —OR″—, —OCOR″— and —COOR″—radicals, wherein R″ is chosen from linear and branched C₁-C₂₀ alkyl radicals optionally comprising at least one hetero atom, for instance oxygen.

For example, the radicals R, R₁, R₂, R₃, and R₄, which may be identical or different, may be chosen from hydrogen atoms, R′, —OR′, —OCOR′ and —COOR′ radicals, wherein R′ is chosen from linear and branched C₁-C₆ alkyl radicals, and from the following optionally neutralized solubilizing groups: —COOH, —CH₂COOH, —CH₂OH, —(CH₂)₆OH, —(CH₂)₃SO₃H, —O(CH₂)₃SO₃H, —O(CH₂)₃N(C H₂CH₃)₂, —[(CH₂)₂O]ₓCH₂CH₂OH, and —[(CH₂)₂O]ₓCH₂CH₂OCH₃, wherein x is an average number ranging from 0 to 200.

The number n of repeating units in the polymer ranges from 5 to 10,000, for instance from 5 to 1,000, such as from 10 to 1,000 and from 20 to 700.

For example, in one embodiment of the present disclosure, the at least one conductive polymer is such that at least one radical of R, R₁, R₂, R₃, and R₄ is a solubilizing group.

In accordance with another embodiment of the present disclosure, the at least one conductive polymer comprises at least one solubilizing group per repeating unit.

In yet another embodiment of the present disclosure, the at least one conductive polymer is soluble in the medium of the composition.

The conductive polymers that may be present in the composition as disclosed herein are known to those skilled in the art and are described, for example, in the book “Handbook of Organic Conductive Molecules and Polymers”-Wiley 1997-New York, Vol 1, 2, 3, and also in the review Can. J. Chem. Vol. 64, 1986.

Polythiophenes and their synthesis are described, for instance, in the article taken from the review Chem. Mater. 1998, Vol. 10, No 7, pages 1990-1999 by the authors Rasmussen S. C., Pickens J. C. and Hutchison J. E. “A new, general approach to tuning the properties of functionalized polythiophenes: The oxidative polymerization of monosubstituted bithiophenes;” in the article taken from the review Macromolecules, 1998, 31, pages 933-936, by the same authors, and “Highly conjugated, water-soluble polymers via direct oxidative polymerization of monosubstituted bithiophenes.” In addition to polymerization via chemical or electrochemical oxidation, the polymers may also be obtained by polycondensation (dihalothiophene; catalysis with nickel or palladium complexes); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond in the presence of a Pd-based catalyst —AA-BB or A-B type); via Reike polymerization (organozinc in the presence of a nickel complex); via polymerization of McCulloch type, and so on.

As a non-limiting example, the conductive polymers that may be used in the composition according to the present disclosure are described in international patent application WO 99/47570.

Further among the conductive polymers that are suitable for use according to the present disclosure, non-limiting mention may be made for example, of the polymers of formulae (IIIa), (IIIb) and (IIIc) wherein the solubilizing groups are for instance, chosen from carboxylic acid groups; sulfonic acid groups; tertiary amine radicals; quaternary ammonium radicals such as —NR′₃⁺Z⁻ wherein Z is chosen from Br and Cl atoms, and (C₁-C₄)alkyl-OSO₃ radicals, and R′, which may be identical or different, are chosen from linear and branched C₁ to C₂₀ alkyls, or two R′ may form a heterocycle with the nitrogen; the solubilizing groups may optionally be connected to the ring via a spacer; and the carboxylic and sulfonic acid functional groups may optionally be neutralized.

Thus, the polymerization may be performed via chemical or electrochemical oxidation of the corresponding thiophene monomer or else via polycondensation.

By way of non-limiting illustration, the polythiophenes of formulae (IIIa) and (IIIb) may be obtained by polymerization via oxidation (for example with FeCl₃ catalysis); via polycondensation of dihalothiophene catalyzed with nickel or palladium complexes (e.g.: NiCl₂(dppe)₂); via Suzuki coupling (coupling between a halogen function, for example bromine, and a boronic acid, catalysis: palladium complex and base; this then gives coupling of M-BB type (reaction of monomers of the type A-X-A with B-X′-B) or of A-B type (reaction of several monomers of the type A-X-B); via Stille coupling (formation of a carbon-carbon bond formed in the presence of a Pd-based catalyst —AA-BB or A-B type); via Reike polymerization (organozinc in the presence of a nickel complex); via polymerization of McCulloch type, and so forth.

By way of non-limiting example, the vinylene polythiophenes of formula (IIIc), wherein Z is a —CH═CH— radical, may be obtained, for instance, via Gilch polymerization in the presence of a strong base (potassium tert-butoxide) of 2,5-bis(bromoalkylene)thiophene; via polymerization by the Wessling method via the use of a precursor based on sulfonium salts and pyrolysis; and via a Wiftig-Horner Wittig reaction.

Further by way of non-limiting example, the ethynylene polythiophenes of formula (IIIc) wherein Z is a —C≡C—, may be obtained by Heck-Sonogashira coupling (of the type AA-BB or A-B; formation of a carbon-carbon bond between a terminal acetylenic (or true acetylenic) functional group and a bromo or iodo functional group, catalyzed with a palladium/copper complex (PdCl₂(PPh₃)₃, CuI or Cu(OAc)₂) in the presence of a base such as triethylamine, diisopropyl amine, piperidine, etc.); and via metathesis of alkynes in the presence of a molybdenum complex (Mo(CO)₆).

In most cases, the functionalization, i.e., the introduction of the solubilizing or non-solubilizing group(s) of the polythiophenes, is performed on the monomer before it is polymerized. In certain cases, the solubilizing group is obtained after working up the polymer. This is the case, for instance, for the carboxylic acid functional group, which may be obtained by hydrolysis of the corresponding ester.

For example, the solubilizing groups may be chosen from carboxylic acid groups; sulfonic acid groups; tertiary amine radicals; quaternary ammonium radicals such as —NR′₃⁺Z⁻ wherein Z is chosen from Br and Cl atoms, and (C₁-C₄)alkyl-OSO₃ radicals, wherein R′, which may be identical or different, are chosen from linear and branched C₁-C₂₀ alkyl radicals, optionally connected to the ring via a spacer, such as C₁-C₂₀ alkyl radicals; and the salts thereof. The carboxylic and sulfonic acid functional groups may optionally be neutralized.

According to one embodiment of the present disclosure, for instance, the conductive polymer is chosen from formulae (IIIa), (IIIb) and (IIIc), wherein at least one radical chosen from R₁, R₂, R₃, and R₄ of formula (IIIa), or chosen from R₁ and R₂ of formulae (IIIb) and (IIIc), is a solubilizing group of carboxylic acid type, in neutralized or non-neutralized form, optionally connected to the ring via a spacer, for instance, chosen from linear and branched C₁-C₂₀ alkyl radicals, wherein the other radical(s) are chosen from hydrogen atoms.

The conductive polymers are generally present in the composition in an amount of at least 0.001% by weight, for example at least 0.01% by weight, such as at least 0.1% by weight and at least 0.5% by weight, relative to the total weight of the composition. Moreover, the amount of conductive polymer may be, for instance, up to 50% by weight, such as up to 30% by weight, up to 20% by weight, and for further example up to 10% by weight, relative to the total weight of the composition.

According to another embodiment of the present disclosure, the amount of conductive polymer ranges from 0.1% to 50% by weight, for instance, from 0.1% to 30% by weight, such as from 0.5% to 10% by weight, relative to the total weight of the composition.

As disclosed above, the composition according to the present disclosure comprises at least one propellant. It is possible for the propellant to be soluble or insoluble, in the composition.

For example, the propellant may be chosen from C₃-C₅ hydrocarbons, such as n-butane, propane, isobutane or pentane; fluoro or chloro hydrocarbons; carbon dioxide; nitrous oxide; dimethyl ether; nitrogen; and compressed air. Mixtures of propellants may also be used.

In one embodiment of the present disclosure, dimethyl ether is used as the propellant.

The propellant amount can range, for example, from 5% to 90% by weight, relative to the total weight of the composition in the aerosol device, such as, from 10% to 60% by weight, relative to the total weight of the composition in the aerosol device.

The cosmetically acceptable medium of the composition comprises water or a mixture of water and at least one organic solvent chosen from those that are acceptable in the field.

Among the organic solvents that may be used, non-limiting mention may be made more, for example, of C₁-C₄ alcohols, such as ethyl alcohol and isopropyl alcohol, aromatic alcohols, for instance benzyl alcohol and phenylethyl alcohol, or glycols or glycol ethers, for instance ethylene glycol monomethyl ether, monoethyl ether and monobutyl ether, propylene glycol or its ethers, for instance propylene glycol monomethyl ether, butylene glycol, dipropylene glycol and also diethylene glycol alkyl ethers, for instance diethylene glycol monoethyl ether or monobutyl ether, or polyols, for instance glycerol; further, polyethylene glycols and polypropylene glycols, and mixtures of all these compounds, may also be used.

A person skilled in the art can determine without difficulty the appropriate composition of the solvent present in the composition. However, for example, the solvent may comprise at least 50% by volume of C₂-C₄ alcohol, such as at least 70% by volume of C₂-C₄ alcohol.

The composition as disclosed herein is packaged in a suitable aerosol container placed under pressure using the abovementioned propellant.

The composition may furthermore comprise at least one direct dye. For example, the at least one direct dye can be chosen from nonionic, cationic and anionic direct dyes. In general, the at least one direct dye can be chosen from nitrobenzene dyes, azo, azomethine, methine, anthraquinone, naphthoquinone, benzoquinone, phenothiazine, indigoid, xanthene, phenanthridine, phthalocyanin and triarylmethane-based dyes, natural dyes (for instance henna or camomile), and mixtures thereof.

When they are present, the at least one direct dye can be present in an amount, for example, ranging from 0.0005% to 12% by weight, relative to the total weight of the composition, such as ranging from 0.005% to 6% by weight, relative to the total weight of the composition.

The composition according to the present disclosure may also comprise at least one surfactant, which may be chosen from anionic, amphoteric, nonionic, zwitterionic and cationic surfactants, and mixtures thereof.

Among the surfactants that may be used, non-limiting mention may be made of alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters and other nonionic surfactants of the hydroxypropyl ether type.

When the composition comprises at least one surfactant, it can present in an amount less than 30% by weight, such as ranging from 0.5% to 10% by weight, relative to the total weight of the composition.

The cosmetic composition may also comprise an effective amount of other agents known for use in the treatment of human keratin fibers, such as fixing polymers, thickeners, film-forming polymers, styling polymers, antioxidants, fragrances, dispersants, conditioners, for instance, cationic and amphoteric polymers, opacifiers, UV-screening agents, preserving agents, ceramides, pseudoceramides, vitamins or provitamins, for instance panthenol, and nonionic, anionic, amphoteric or cationic associative polymers.

Needless to say, a person skilled in the art will take care to select the optional additional compound(s) mentioned above such that the beneficial properties intrinsically associated with the dye composition according to the invention are not, or are not substantially, adversely affected by the envisaged addition(s).

The composition according to the present disclosure is packaged so as to obtain a lacquer or a mousse.

The composition as disclosed herein may applied to wet or dry keratin fibers, and the fibers may be then dried or left to air dry. For example, the fibers may be shaped at the time of drying.

The drying operation can take place within a temperature range ranging from 20 to 120° C., such as from 20 to 80° C.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The example that follows illustrates the present disclosure without, however, being limiting in nature.

### EXAMPLE

### Synthesis of poly(thiophene-3-acetic acid)

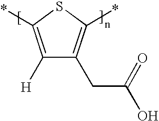

Procedure

Preparation of the polymer: poly(ethyl thiophene-3-acetate)

25 ml of dry chloroform were introduced into a Schienk tube under argon, the system was degassed and the following reagents were then introduced:
2.5 g of ethyl thiophene-3-acetate (14.7 mmol) 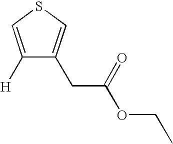
and 1 g of FeCl₃ (6.15 mmol).

The mixture was stirred for 24 hours under argon at 50° C.

The poly(ethyl thiophene-3-acetate) polymer was then precipitated in heptane.

The polymer was then dissolved in a tetrahydrofuran solution.

Infrared Characterization:
C═O band: 1719 cm⁻¹; CH₂, CH₃ bands=2979 cm⁻¹, 2934 cm⁻¹ and disappearance of the CH band at 3102 cm⁻¹ were present in the monomer.

Hydrolysis of the Polymer: poly(ethyl thiophene-3-acetate) to form poly(thiophene-3-acetic acid)

The polymer obtained above was then hydrolysed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was filtered off and washed several times with distilled water in order to remove the traces of catalyst.

Infrared Characterization of the Polymer:
C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation comprising the Polymer and process using it:

| | |
|---|---|
| Poly(thiophene-3-acetic acid) | 1 g |
| Aminomethyl propanol qs | pH 7 |
| Water qs | 50 g |
| Ethyl alcohol | 15 g |
| Dimethyl ether | 35 g |

The can was equipped with a valve fitted with a dip tube and a pushbutton fitted with a nozzle.

The composition was vaporized about 20 cm away from the hair for about 5 seconds, and gave the hair a sheen effect.

1. A composition comprising, in a cosmetically acceptable medium:
at least one propellant and
at least one conductive polymer.

2. The composition according to claim 1, wherein the at least one conductive polymer comprises at least one repeating unit chosen from:
anilines of formula (I): 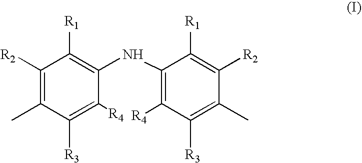
pyrroles of formulae (IIa) and (IIb): 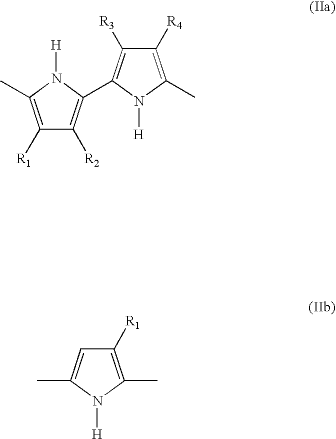
thiophenes and bisthiophenes of formulae (IIIa), (IIIb) and (IIIc): 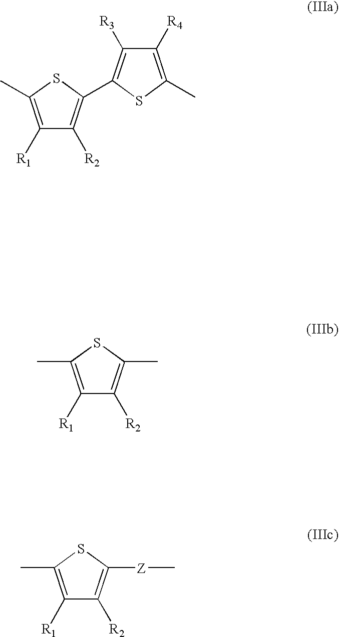
furans of formula (IV) 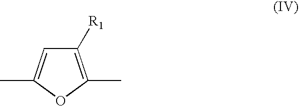
para-phenylene sulfides of formula (V) 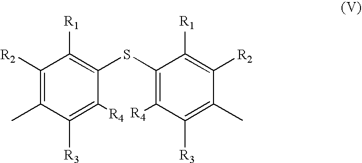
para-phenylenevinylenes of formula (VI): 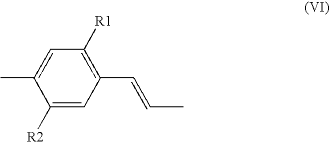
indoles of formula (VII): 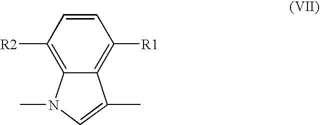
aromatic amides of formulae (VIIIa), (VIIIb), (VIIIc) and (VIIId): 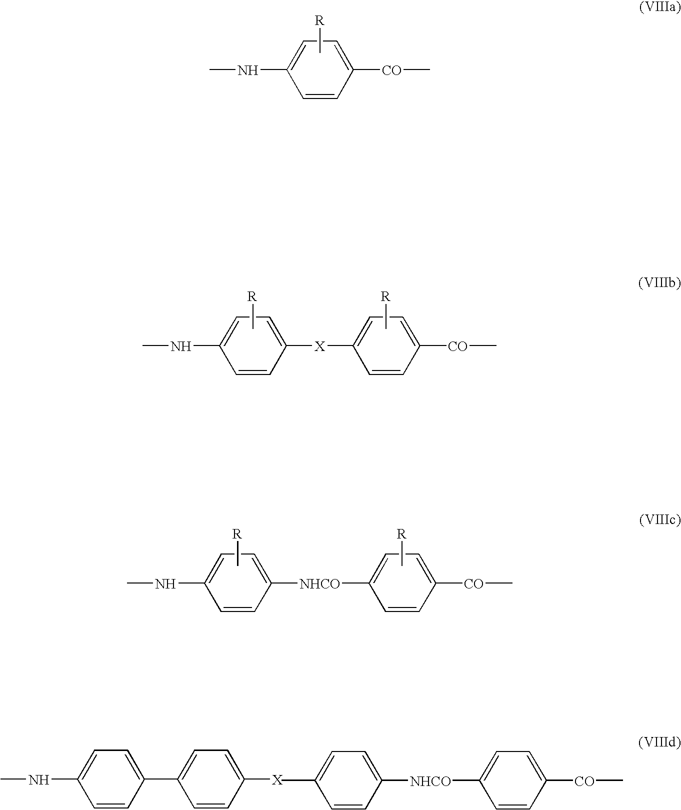
aromatic hydrazides of formulae (IXa), (IXb) and (IXc): 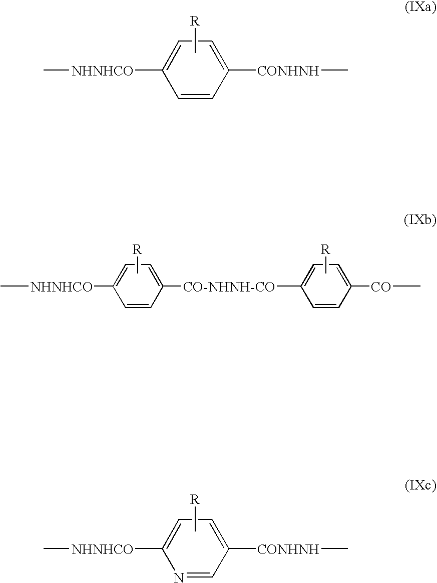
aromatic azomethines of formulae (Xa), (Xb) and (Xc): 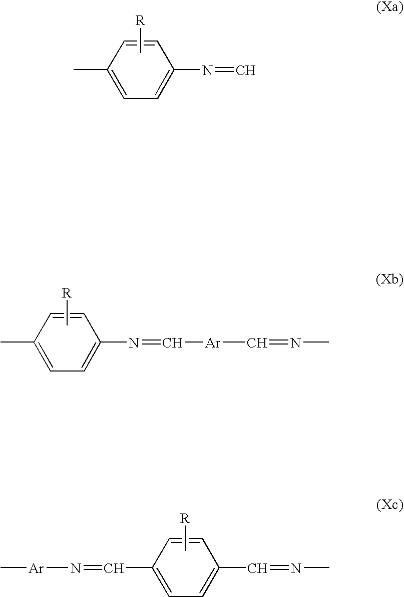
aromatic esters of formulae (XIa), (XIb) and (XIc): 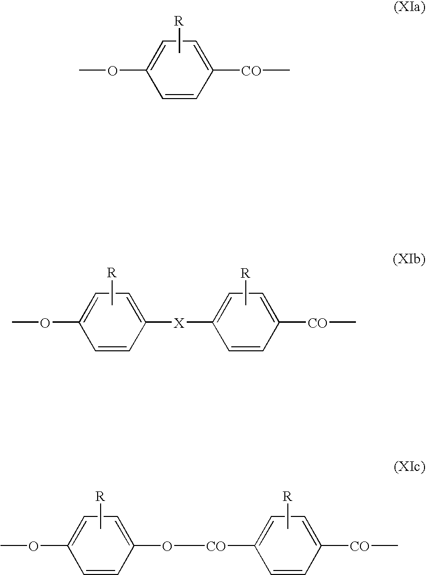
wherein, in formulae (I) to (XI):
R, R₁, R₂, R₃, and R₄, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring, wherein at least one radical chosen from R, R₁, R₂, R₃, and R₄, is a solubilizing group;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

3. The composition according to claim 2, wherein the at least one solubilizing group is chosen from:
—COOH and —COO⁻M⁺ radicals, wherein M is chosen from alkali metals, alkaline-earth metals, organic amines, alkanolamines, and amino acids,
-SO₃H and —SO₃⁻M⁺ radicals, wherein M is chosen from alkali metals, alkaline-earth metals, organic amines, alkanolamines, and amino acids,
primary, secondary and tertiary amine radicals,
quaternary ammonium radicals,
hydroxyl radicals, and
poly((C₂-C₃)alkylene oxide) radicals.

4. The composition according to claim 3, wherein the quaternary ammonium radicals are —NR₁₃⁺Z⁻, wherein Z is chosen from Br and Cl atoms, and (C₁-C₄)alkyl-OSO₃ radicals, and wherein R′, which may be identical or different, is chosen from linear and branched C₁ to C₂₋₀ alkyl radicals, or two R′ may form a heterocycle with the nitrogen.

5. The composition according to claim 2, wherein the at least one solubilizing group is connected to the ring via a spacer group.

6. The composition according to claim 5, wherein the spacer group is chosen from, —R″—, —OR″—, —OCOR″— and —COOR″— radicals, wherein R″ is chosen from linear and branched C₁-C₂₀ alkyl radicals optionally comprising at least one hetero atom.

7. The composition according to claim 2, wherein R, R₁, R₂, R₃, and R₄, which may be identical or different, are chosen from hydrogen atoms, and R′, —OR′, —OCOR′ and —COOR′ radicals, wherein R′ is chosen from linear and branched C₁-C₆ alkyl radicals, and from the optionally neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

8. The composition according to claim 1, wherein the at least one conductive polymer is in a soluble form in the medium.

9. The composition according to claim 1, wherein the at least one conductive polymer comprises at least one solubilizing group per repeating unit.

10. The composition according to claim 3, wherein the at least one solubilizing group is chosen from carboxylic acid groups; sulfonic acid groups; tertiary amine radicals; quaternary ammonium radicals; and the salts thereof; wherein the at least one solubilizing group is optionally connected to the ring via a spacer; and further wherein the carboxylic and sulfonic acid functional groups may optionally be neutralized.

11. The composition according to claim 10, wherein the quaternary ammonium radicals are —NR′₃⁺Z⁻ radicals wherein Z is chosen from Br and Cl atoms and (C₁-C₄)alkyl-OSO₃ radicals, and wherein R′, which may be identical or different, are chosen from linear and branched C₁-C₂₀ alkyl radicals.

12. The composition according to claim 10, wherein the at least one solubilizing group is connected to the ring via a spacer chosen from C₁-C₂₀ alkyl radicals.

13. The composition according to claim 2, wherein the at least one conductive polymer is chosen from polymers comprising at least one repeating unit of formulae (IIIa), (IIIb) and (IIIc), wherein at least one radical chosen from R₁, R₂, R₃, and R₄ of formula (lla) or R₁ and R₂ of formulae (IIIb) and (IIIc) is an optionally neutralized carboxylic acid solubilizing group, optionally connected to the ring via a spacer, and wherein the other radicals are hydrogen atoms.

14. The composition according to claim 13, wherein the at least one solubilizing group is connected to the ring via a spacer chosen from linear and branched C₁-C₂₀ alkyl radicals.

15. The composition according to claim 1, wherein the at least one conductive polymer is present in an amount of at least 0.001% by weight, relative to the total weight of the composition.

16. The composition according to claim 1, wherein the at least one conductive polymer is present in an amount of up to 50% by weight, relative to the total weight of the composition.

17. The composition according to claim 1, wherein the at least one conductive polymer is present in an amount ranging from 0.1% to 50% by weight, relative to the total weight of the composition.

18. The composition according to claim 1-, wherein the at least one conductive polymer has a conductivity ranging from 10⁻⁵ to 5×10⁻⁵ siemens/cm.

19. The composition according to claim 1, wherein the at least one propellant is chosen from C₃-C₅ hydrocarbons, fluoro and chloro hydrocarbons, carbon dioxide, nitrous oxide, dimethyl ether, nitrogen and compressed air.

20. The composition according to claim 19, wherein the at least one propellant is present in an amount ranging from 5% to 90% by weight, relative to the total weight of the composition in the aerosol device.

21. The composition according to claim 20, wherein the at least one propellant is present in an amount ranging from 10% to 60% by weight, relative to the total weight of the composition in the aerosol device.

22. The composition according to claim 1, wherein the cosmetically acceptable medium is water or a mixture of water with at least one organic solvent.

23. The composition according to claim 22, wherein the at least one organic solvent is chosen from alcohols, glycols, glycol ethers, polyols, polyol ethers, polyethylene glycols, and polypropylene glycol.

24. The composition according to claim 23, wherein the solvent comprises at least 50% by volume of C₂-C₄ alcohol.

25. The composition according to claim 24, wherein the solvent comprises at least 70% by volume of C₂-C₄ alcohol.

26. The composition according to claim 1, further comprising at least one direct dye.

27. The composition according to claim 26, wherein the at least one direct dye is chosen from nonionic, cationic and anionic direct dyes.

28. The composition according to claim 26, wherein the at least one direct dye is present in an amount ranging from 0.0005% to 12% by weight relative to the weight of the dye composition.

29. The composition according to claim 1, further comprising at least one surfactant, wherein the at least one surfactant is chosen from anionic, amphoteric, nonionic, zwitterionic and cationic surfactants.

30. A process for treating human keratin fibers comprising applying a composition comprising, in a cosmetically acceptable medium:
at least one propellant and
at least one conductive polymer
to wet or dry keratin fibers, and either drying the fibers thus treated or leaving them to air dry.

31. The process according to claim 30, wherein the human keratin fibers are hair.

32. The process according to claim 30, wherein the at least one conductive polymer comprises at least one repeating unit chosen from:
anilines of formula (I): 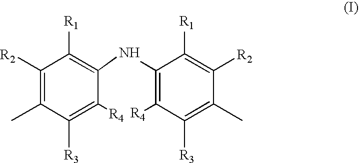
pyrroles of formulae (IIa) and (IIb): 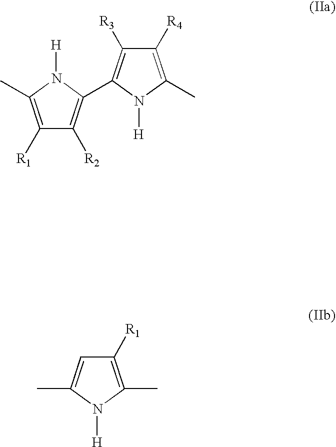
thiophenes and bisthiophenes of formulae (IIIa), (IIIb) and (IIIc): 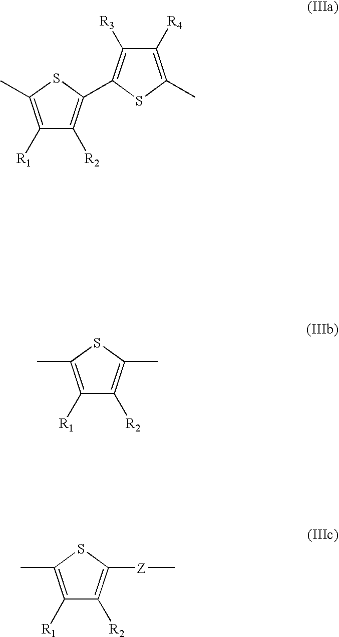
furans of formula (IV): 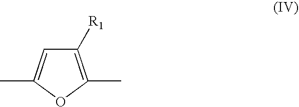
para-phenylene sulfides of formula (V): 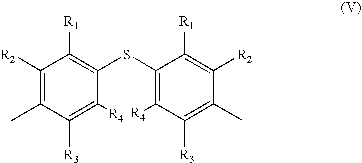
para-phenylenevinylenes of formula (VI): 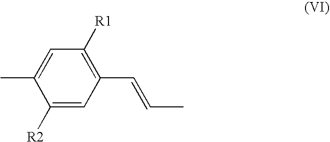
indoles of formula (VII): 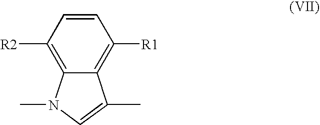
aromatic amides of formulae (VIIIa), (VIIIb), (VIIIc) and (VIIId): 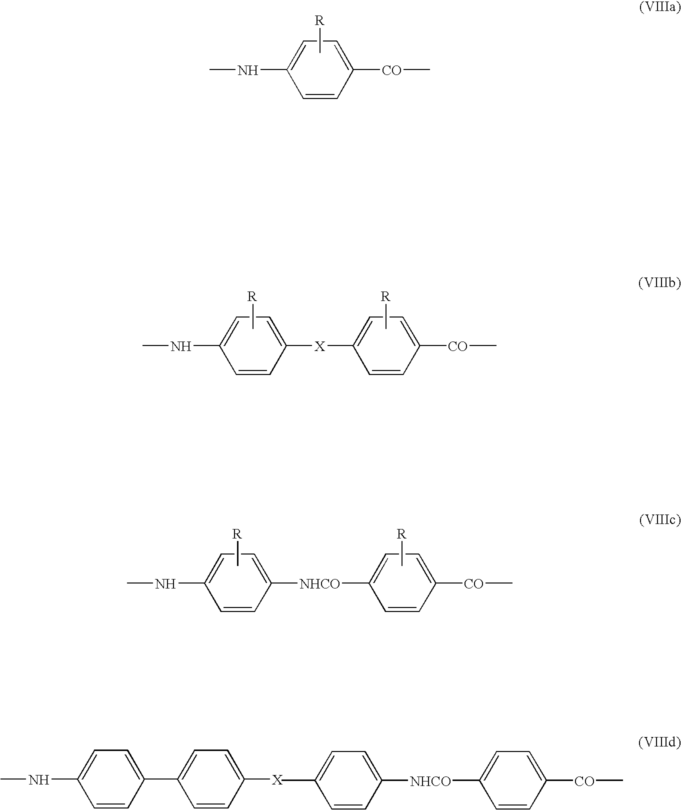
aromatic hydrazides of formulae (IXa), (IXb) and (lXc): 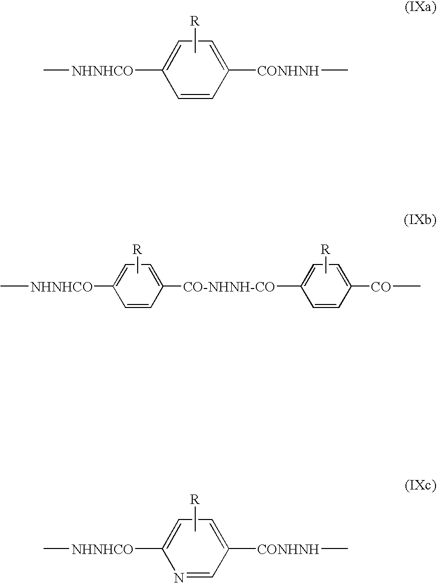
aromatic azomethines of formulae (Xa), (Xb) and (Xc): 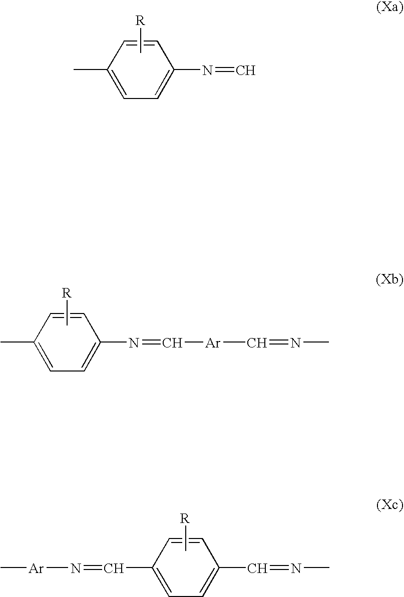
aromatic esters of formulae (XIa), (XIb) and (XIc): 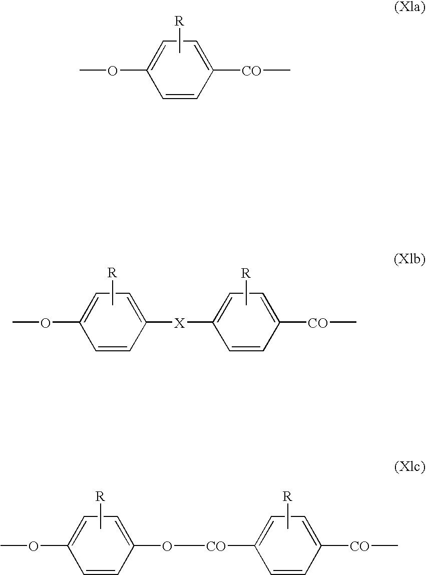
wherein, in formulae (I) to (XI):
R, R₁, R₂, R₃, and R₄, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring, wherein at least one radical chosen from R, R₁, R₂, R₃, and R₄, is a solubilizing group;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

33. The process according to claim 32, wherein the at least one solubilizing group is chosen from:
—COOH and —COO⁻M⁺ radicals, wherein M is chosen from alkali metals, alkaline-earth metals, organic amines, alkanolamines, and amino acids,
—SO₃H and —SO₃⁻M⁺ radicals, wherein M is chosen from alkali metals, alkaline-earth metals, organic amines, alkanolamines, and amino acids,
primary, secondary and tertiary amine radicals,
quaternary ammonium radicals,
hydroxyl radicals, and
poly((C₂-C₃)alkylene oxide) radicals.

34. The process according to claim 33, wherein the quaternary ammonium radicals are —NR′₃⁺Z⁻, wherein Z is chosen from Br and Cl atoms, and (C₁-C₄)alkyl-OSO₃ radicals, and wherein R′, which may be identical or different, is chosen from linear and branched C₁ to C₂₀ alkyl radicals, or two R′ may form a heterocycle with the nitrogen.

35. The process according to claim 33, wherein the at least one solubilizing group is connected to the ring via a spacer group.

36. The process according to claim 35, wherein the spacer group is chosen from, —R″—, —OR″—, —OCOR″— and —COOR″— radicals, wherein R″ is chosen from linear and branched C₁-C₂₀ alkyl radicals optionally comprising at least one hetero atom.

37. A method for cosmetically treating keratin fibers comprising, applying to the keratin fibers a composition comprising, in a cosmetically acceptable medium, at least one conductive polymer and at least one propellant, wherein the at least one conductive polymer and the at least one propellant are present in an amount sufficient to give the keratin fibers an optical effect.

38. The method according to claim 37, wherein the at least one conductive polymer comprises at least one repeating unit chosen from:
anilines of formula (1): 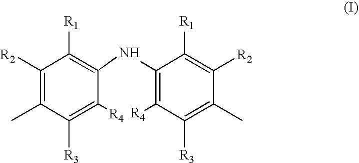
pyrroles of formulae (IIa) and (IIb): 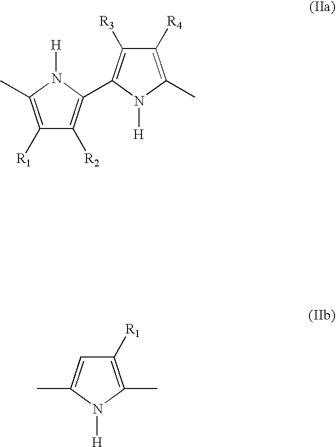
thiophenes and bisthiophenes of formulae (IIIa), (IIIb) and (IIIc): 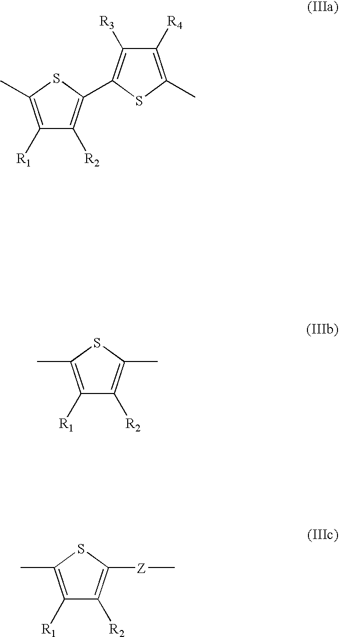
furans of formula (IV): 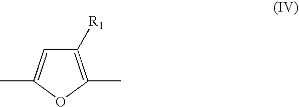
para-phenylene sulfides of formula (V): 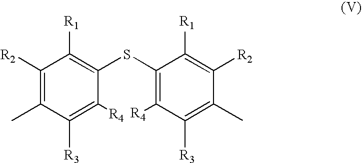
para-phenylenevinylenes of formula (VI): 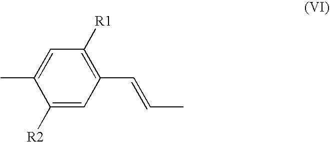
indoles of formula (VII): 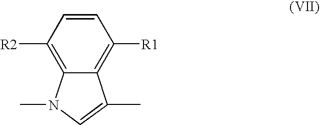
aromatic amides of formulae (VIIIa), (VIIIb), (VIIIc) and (VIIId): 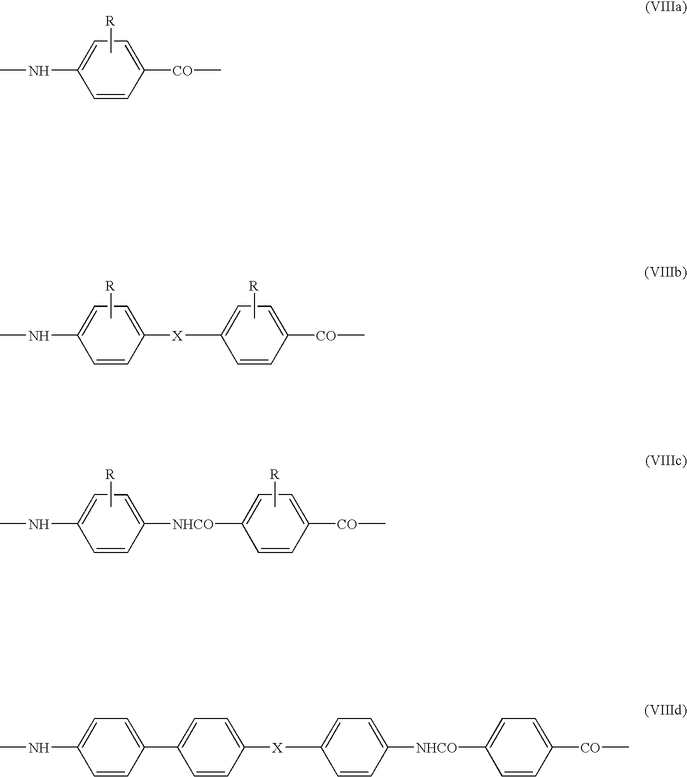
aromatic hydrazides of formulae (IXa), (IXb) and (IXc): 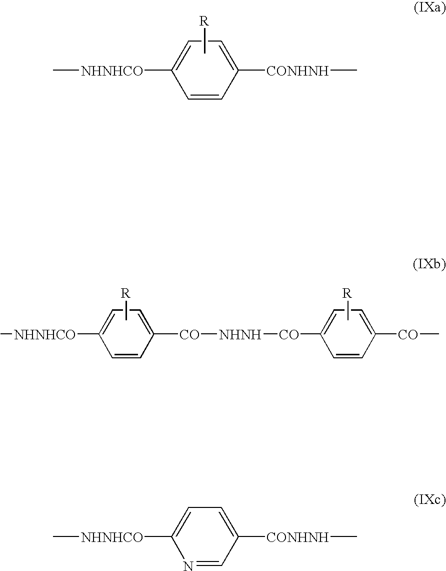
aromatic azomethines of formulae (Xa), (Xb) and (Xc): 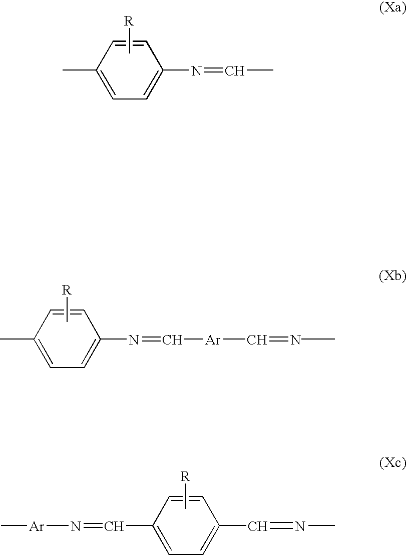
aromatic esters of formulae (XIa), (XIb) and (XIc): 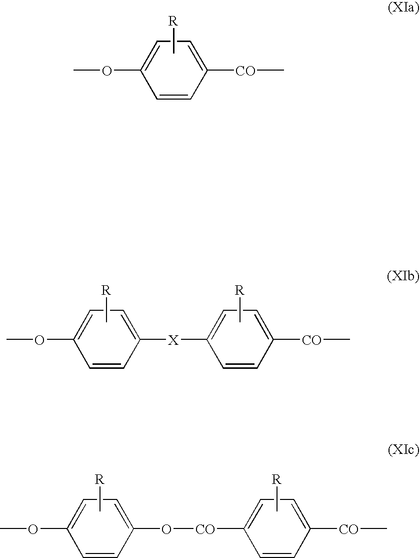
wherein, in formulae (I) to (XI):
R, R₁, R₂, R₃, and R₄, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring, wherein at least one radical chosen from R, R₁, R₂, R₃, and R₄, is a solubilizing group;
Ar is a radical comprising a monoaromatic or polyaromatic radical;
X is chosen from oxygen and sulfur atoms, —NHCO—, —SO₂—, —N═N—, —C(CH₃)₂—, —CH₂—, —CH═CH—, and —CH═N— radicals; and
Z is chosen from —CH═CH— and —C≡C— radicals.

39. The method according to claim 37, wherein the optical effect is a sheen effect.

40. A device comprising a cosmetic composition comprising, in a cosmetically acceptable medium:
at least one propellant and
at least one conductive polymer.
